(19) United States
(12) Patent Application Publication
(10) Pub. No.: US 2026/0355460 A1
(43) Pub. Date: Nov. 12, 2026

(54) CONDITIONAL BRAKE ENGAGEMENT TO INHIBIT BACK-DRIVING OF SYSTEM COMPONENTS

(71) Applicant: Intuitive Surgical Operations, Inc., Sunnyvale, CA (US)

(72) Inventors: Sang Gyum Kim, Sunnyvale, CA (US); Samuel B. Schorr, Sunnyvale, CA (US); Shibing Liu, Sunnyvale, CA (US)

(73) Assignee: Intuitive Surgical Operations, Inc., Sunnyvale, CA (US)

(21) Appl. No.: 19/515,145

(22) PCT Filed: Aug. 29, 2024
(86) PCT No.: PCT/US2024/044405

§ 371 Date: Feb. 27, 2026

Related U.S. Application Data
(60) Provisional application No. 63/535,922, filed on Aug. 31, 2023.

(51) Int. Cl. A61M 25/01; A61B 1/00; A61B 90/00; F16D 59/00; F16D 121/22

(52) U.S. Cl. CPC A61M 25/0113; A61B 1/0016; A61B 90/03; A61B 2090/0805; F16D 59/00; F16D 2121/22

(57) ABSTRACT

A medical system includes a manipulator assembly and a control system coupled to the manipulator assembly. The manipulator assembly includes an actuator for driving a medical instrument along an insertion axis of the manipulator assembly and a brake for inhibiting movement of the medical instrument along the insertion axis. The control system is configured to determine whether the medical system is in a first operation mode involving actuator driven movement of the medical instrument along the insertion axis or a second operation mode involving no actuator-driven movement of the medical instrument along the insertion axis, and based determining that the medical system is in the second operation mode, control the brake to inhibit movement of the medical instrument along the insertion axis caused by an external force.

### CROSS-REFERENCE TO RELATED APPLICATIONS

This application claims the benefit of priority under 35 U S.C. § 119(e) to U.S. Provisional Patent Application Ser. No. 63/535,922 filed on Aug. 31, 2023, which is hereby incorporated by reference herein in its entirety.

### FIELD

Disclosed embodiments relate to improved robotic and/or medical devices, systems, and methods.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter or bronchoscope, that can be inserted into anatomic passageways and navigated toward a region of interest within the patient anatomy.

Under some operating conditions, a medical instrument may be driven along one or more movement axes, and under other operating conditions, the medial instrument is not driven, i.e., kept stationary. In this case, an unintentional back-driving by external forces acting on the medical system may cause undesirable movement of the medical instrument.

### SUMMARY

The following presents a simplified summary of various examples described herein and is not intended to identify key or critical elements or to delineate the scope of the claims.

In some examples, a medical system comprises: a manipulator assembly comprising: an actuator for driving a medical instrument along an insertion axis of the manipulator assembly; and a brake for inhibiting movement of the medical instrument along the insertion axis; and a control system coupled to the manipulator assembly, the control system configured to: determine whether the medical system is in a first operation mode involving actuator driven movement of the medical instrument along the insertion axis or a second operation mode involving no actuator-driven movement of the medical instrument along the insertion axis; and based determining that the medical system is in the second operation mode, control the brake to inhibit movement of the medical instrument along the insertion axis caused by an external force.

In some examples, a non-transitory machine-readable medium comprises a plurality of machine-readable instructions executed by one or more processors associated with a medical system, the plurality of machine-readable instructions causing the one or more processors to perform a method comprising: determining whether the medical system is in a first operation mode involving actuator driven movement of a medical instrument along an insertion axis or a second operation mode involving no actuator-driven movement of the medical instrument along the insertion axis; and based determining that the medical system is in the second operation mode, controlling a brake to inhibit movement of the medical instrument along the insertion axis caused by an external force.

In some examples, a method for operating a medical system comprises: determining whether the medical system is in a first operation mode involving actuator-driven movement of a medical instrument along an insertion axis or a second operation mode involving no actuator driven movement of the medical instrument along the insertion axis; and based determining that the medical system is in the second operation mode, controlling a brake to inhibit movement of the medical instrument along the insertion axis caused by an external force.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within a flexible elongate device according to some embodiments.

FIG. 2C is a simplified perspective diagram of a manipulator assembly including an instrument and a manipulator arm holding the instrument according to some embodiments.

FIG. 2D shows a cross-sectional view of an actuator-brake assembly according to some embodiments.

FIGS. 3A and 3B are simplified diagrams of side views of a patient coordinate space including a medical instrument mounted on an insertion assembly according to some embodiments.

FIG. 4 is a simplified perspective diagram of an input control console according to some embodiments.

FIG. 5 is an illustration of operation modes of a medical system according to some embodiments.

FIG. 6 is a flowchart of a method according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Embodiments of the disclosure include medical systems and methods for operating such medical systems. Medical systems may be medical systems that use flexible elongate devices (e.g., catheters, bronchoscopes, endoscopes, etc.), but also other medical systems.

A medical system may include a medical instrument, and the medical instrument may be driven along one or more movement axes. In some embodiments, the driving may involve an actuator driven movement of the medical instrument along an insertion axis to move the medical instrument towards or away from target tissue. The driving may occur when the medical system operates in a first operation mode of the medical system. The first operation mode may be associated with, for example, a navigation operation that involves movement of the medical instrument within an anatomical passageway toward the target tissue.

In some embodiments, the medical system may further operate in a second operation mode that involves no actuator driven movement of the medical instrument along the insertion axis. The second operation mode may be associated with, for example, a medical operation conducted on the target tissue. The medical operation may include a biopsy, an ablation, an electroporation, etc. The second operation mode may be associated with a user intent for external force application to the medical instrument which may move the medical instrument along the insertion axis.

In some embodiments, the medical system includes a brake that inhibits movement of the medical instrument along the insertion axis when the brake is engaged. In some embodiments, the brake may be engaged when the medical device is operating in the second operation mode. Embodiments of the disclosure involve engaging the brake when transitioning from the first operation mode to the second operation mode, and releasing the brake when transitioning from the second operation mode to the first operation mode upon detection of such transitions. The brake may remain engaged during the second operation mode and may remain released during the first operation mode. Although transitions between operation modes may be used to control operation of the brake, there may be delays between when operation mode transitions are determined and when brake control is initiated to reduce overuse of the brake and avoid false operation mode transition signals.

Engagement of the brake may inhibit an unintentional back-driving of the medical instrument by external forces while the medical system is in the second operation mode. For example, various operations of the medical system may be triggered based on sensor-based detection of insertion or retraction of the medical instrument. These operations may be intended to trigger on actuator driven movement but may be unintentionally triggered by external forces that cause insertion or retraction. In one example, the medical instrument may be a flexible elongate device (e.g., a catheter, bronchoscope, or endoscope) having an articulable body portion that is relaxed on retraction to prevent damage to patient anatomy. If an external force exerted as part of a medical operation (e.g., a biopsy) causes a retraction, then the flexible elongate device may relax unintentionally, which may result in loss of the intended pose used to perform the medical operation. As such, inhibiting unintentional back-driving of the medical instrument by external forces in the second operation mode can help avoid negatively affecting the medical operation or impairing the performance of medical instruments. Embodiments of the disclosure, thus, rely on a brake in addition to the actuator being servo-controlled to jointly inhibit undesirable and/or unintended movement of the medical instrument. Embodiments of the disclosure include a control system that smoothly engages and releases the brake when transitioning between the first and second operation modes, thereby avoiding disruption of the operations performed by the user operating the medical system. While it would be possible to apply the brake whenever there is no actuator driven insertion or retraction, such a practice can result in excessive brake application that reduces the lifespan of the brake and/or actuator. As such, the control system intelligently determines transitions between the first and second operation modes to perform the brake application only when necessary. For example, the second operation mode may be associated with system and/or procedural states associated with the user being unlikely to intend actuator driven movement and/or the user being likely to apply external force). A discussion of operations likely to be performed in the second operation mode is provided below.

Various information including sensor data and/or user input may be used to determine the change in operation mode in order to control the brake. This sensor data and/or user input can be used to programmatically determine user intent with respect to actuator driven movement of the medical instrument and/or external force application on the medical instrument for transitioning between the first and second operation modes.

A more detailed discussion of the medical system, medical instruments, operation modes of the medical system, the detection of transitions between the operation modes, the control of the brake based on the detection of transitions between the operation modes, back-driving, typical scenarios in which back-driving is more likely to occur, possible consequences of the back-driving, etc. is provided below in reference to the figures.

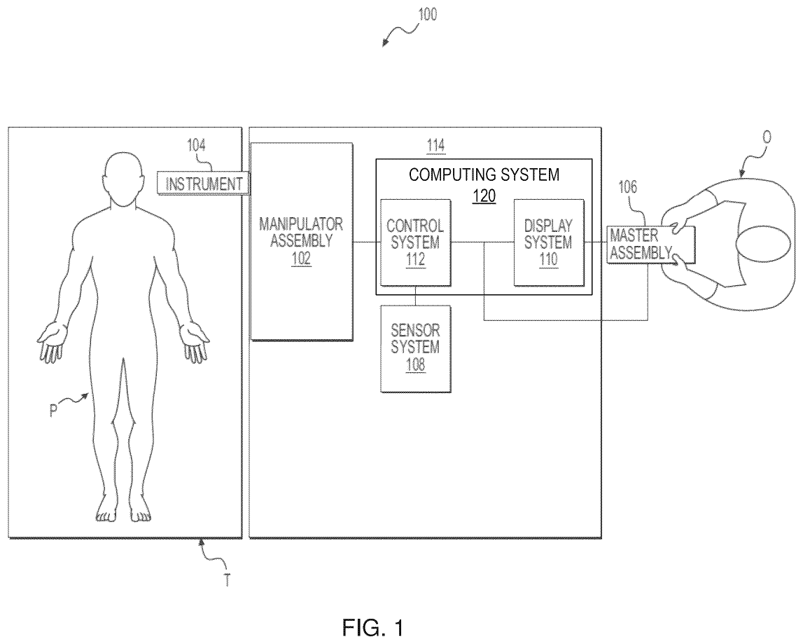

Turning to the figures, FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the medical instrument 104 may be controlled directly by the operator O. In some examples, the manipulator assembly 102 may be manually controlled by the operator O. Direct operator control may include various handles and operator interfaces for hand-held operation of the medical instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo-controlled links (e.g., one or more links that may be manually positioned and locked in place) and/or one or more servo-controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016, and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011, and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

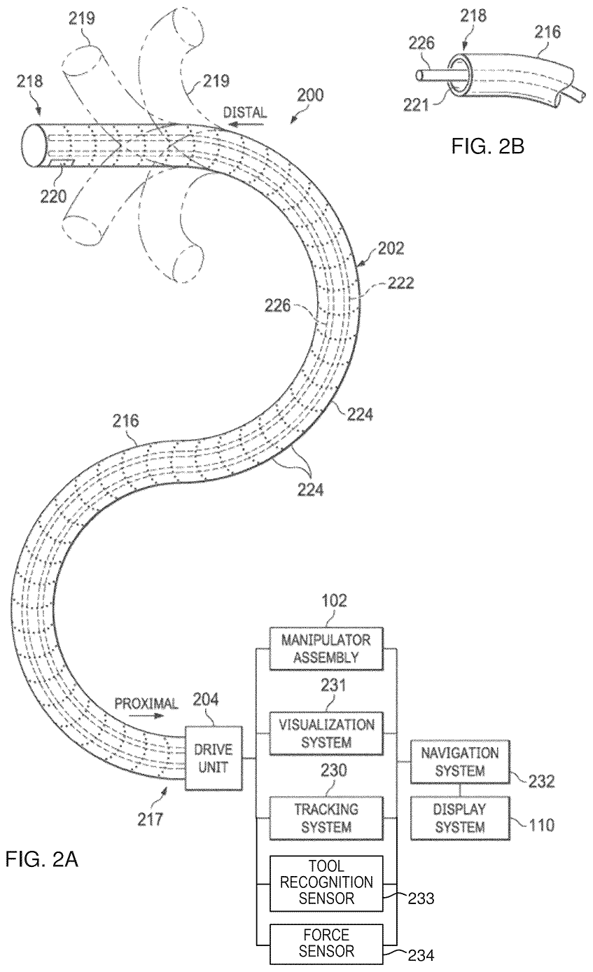

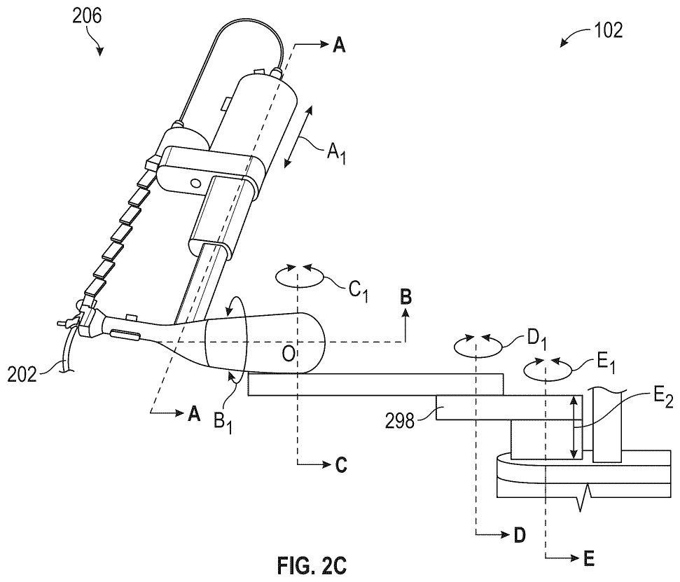

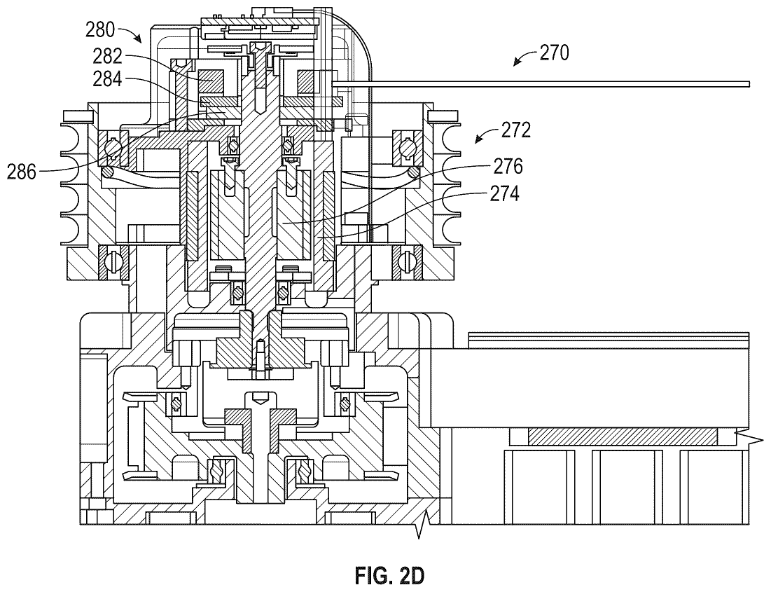

FIG. 2A is a simplified diagram of a medical instrument system 200 according to some embodiments. The medical instrument system 200 includes a flexible elongate device 202 (also referred to as elongate device 202), a drive unit 204, and a flexible tool, e.g., a medical tool, 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, tool recognition sensor 233, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel or lumen 221 through which a flexible tool, e.g., the medical tool, 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexible body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sept. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999, and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor system 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the flexible tool 226 within the elongate device 202 according to some embodiments. The flexible body 216 of the elongate device 202 may include the lumen 221 sized and shaped to receive the flexible tool 226. In some embodiments, the flexible tool 226 may be used for procedures such as diagnostics, imaging, surgery, biopsy, ablation, illumination, irrigation, suction, electroporation, etc. Flexible tool 226 can be deployed through channel or lumen 221 of flexible body 216 and operated at a procedural site within the anatomy. Flexible tool 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the flexible tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The flexible tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the lumen 221 when the biopsy tool is within the lumen 221. The flexible tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of flexible tool 226 to facilitate simultaneous image capture and tissue intervention, such as within the same lumen 221 or in separate channels. A flexible tool 226 may be advanced from the opening of the lumen 221 to perform the procedure (or some other functionality) and then retracted back into the lumen 221 when the procedure is complete. The flexible tool 226 may be removed from the proximal end 217 of the flexible body 216 or from another optional instrument port (not shown) along flexible body 216.

Some embodiments include a tool recognition sensor 233 that may be used to detect the presence, insertion, and/or removal of a flexible tool 226 in the lumen 221. The tool recognition sensor 233 may detect the presence, proximity, and/or absence of targets on the tool to detect insertion signatures for each inserted tool. Accordingly, the tool recognition sensor 233 may also be used to identify tool types (e.g., needles, ablation tools, cutter, graspers, etc.), and based on the recognition of tool type, control mode alternations or tool behavior modifications may be implemented.

The tool recognition sensor 233 can incorporate one or more target readers (not shown) configured to detect one or more targets on a tool and/or catheter. The tool recognition sensor 233 may comprise an inductive sensor (e.g., an inductor or inductive coil that detects a change in inductance caused by ferromagnetic and conductive properties of a material), a capacitive sensor, a Hall effect sensor, a photogate sensor, an optical sensor, a magnetic switch, a barcode scanner, a radio frequency identification (RFID) scanner, a relative position sensor, or combinations thereof that are capable of reading corresponding one or more targets on a tool to be inserted into the lumen 221. Any combination of different types of target readers may be implemented in the tool recognition sensor 233. PCT Publication WO 2020/014207 (published Jan. 16, 2020 and titled “Systems for Sensing Presence of Medical Tools”), which is incorporated by reference herein in its entirety, discloses example systems that include tool recognition sensors.

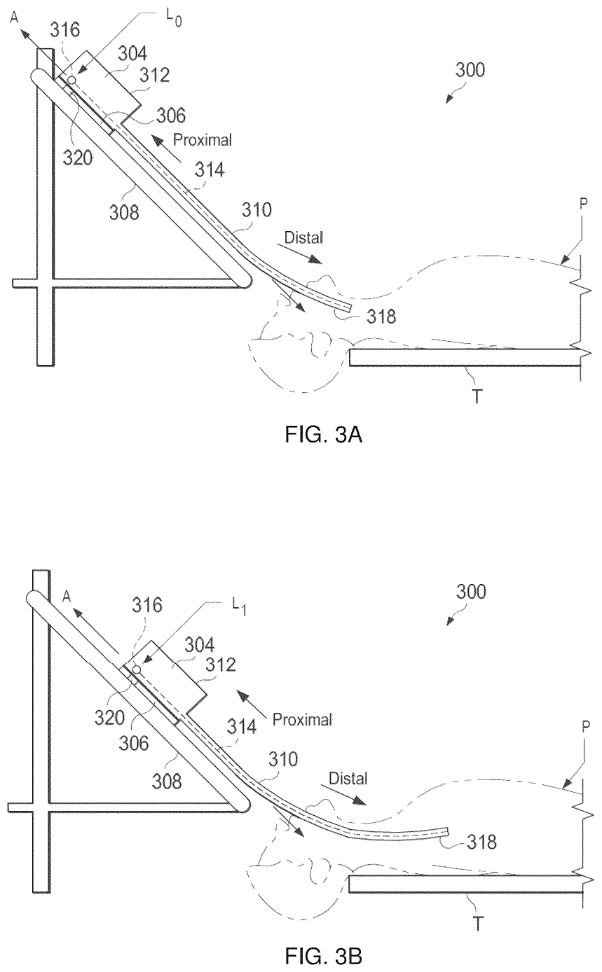

Some embodiments include a force sensor 234. The force sensor 234 may be used to indirectly detect an insertion or removal of a flexible tool 226, based on the external forces involved in the insertion or removal. The force sensor 234 may also detect external forces that are not related to the insertion or removal of a flexible tool. Such external forces may be caused by touch, accidental collisions, etc. The force sensor 234 may be located where external forces associated with the insertion or removal of the flexible tool can be sensed. In some embodiments, the force sensor is mounted on the instrument carriage, describe below in reference to FIGS. 3A and 3B, and may, thus, detect any external force exerted on the instrument carriage or a component attached to the instrument carriage. The force sensor 234 may be able to resolve external forces in different directions, e.g., in an insertion and retraction direction. In some embodiments, the force sensor 234 may be located elsewhere, such as on the flexible elongate device 202.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 215 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005, and titled “Articulated Surgical Instrument for Performing Minimally Invasive Surgery with Enhanced Dexterity and Sensitivity”) and U.S. Pat. No. 9,259,274 (filed Sept. 30, 2008, and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019, and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. The drive unit 204 may further include brakes. One brake may be paired with one actuator. In configurations that pair an actuator with a gear reducer, the brake may be located on the actuator side, which enables even a relatively small brake to produce a significant braking force. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011, and titled “Medical System Providing Dynamic Registration of a Model of an Anatomic Structure for Image-Guided Surgery”), which is incorporated by reference herein in its entirety.

FIG. 2C shows the manipulator assembly 102 including an instrument manipulator 206 coupled to a support structure 298 in accordance with some embodiments of the disclosure. The links of the support structure 298 may include one or more nonservo controlled links (e.g., which may be manually positioned and locked into place) and/or one or more servo-controlled links (e.g., powered links that may be controlled in response to commands from a control system). The support structure 298 provides adjustments to position the instrument manipulator 206 at an optimal position and orientation and/or position the flexible elongate device 202 relative to the patient anatomy or other medical devices. For example, the support structure 298 may provide for a rotation E1 about the axis E, the extension/retraction E2 along the axis E, the rotation D1 about the axis D, and the rotation C1 about the axis C, and the rotation B1, about the axis B, to position the instrument manipulator 206 in a desired position relative to a table (not shown), medical devices (not shown), and/or the patient (not shown).

In some embodiments, optimal location and orientation can include alignment of the instrument manipulator 206 with respect to the patient anatomy, for example, for optimal positioning of the flexible elongate device (202) to minimize friction of the flexible elongate device (202) positioned within the patient anatomy (e.g. anatomical openings, patient vasculature, patient endoluminal passageways, etc.) or within medical devices coupled to patient anatomy (e.g., cannulas, trocars, endotracheal tubes (ETT), laryngeal esophageal masks (LMA), etc.). In other embodiments, optimal location and orientation of the instrument manipulator 206 may additionally or alternatively include optimizing the operator ergonomics by providing sufficient operator workspace and/or ergonomic access to the flexible elongate device 202 when utilizing various medical tools such as needles, graspers, scalpels, grippers, ablation probes, visualization probes, and/or the like, with the flexible elongate device 202.

The instrument manipulator 206 may be further configured to provide teleoperational, robotic control, or other form of controlled translation or manual translation A1 along axis A to provide for insertion and retraction of the flexible elongate device 202 with respect to the patient anatomy.

Each adjustment (e.g., A₁, B₁, C₁, D₁, E₁, and E₂) may be actuated by an actuator that may be combined with a brake. An example is provided in FIG. 2D.

FIG. 2D shows a cross-sectional view of an actuator-brake assembly according to some embodiments. The actuator-brake assembly 270, in the example, is used to control insertion and retraction of the flexible elongate device 202, e.g., along axis A. Similar actuator-brake assemblies may be used to control other degrees of freedom, e.g., along axes B, C, D, and/or E.

The actuator-brake assembly 270 includes a motor 272 and a brake 280. The motor may be any type of motor, e.g., a brushless DC motor with a motor stator 274 and a motor rotor 276. The brake 280 is an electromagnetically controllable brake equipped with a brake coil 282, a brake pressure plate 284 and a brake armature 286. The brake armature 286 may be released by electrically driving the brake coil 282, which causes the brake pressure plate 284 to lift. Other brake designs may be used, without departing from the disclosure.

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308. The instrument carriage 306 or insertion stage 308 may further include brakes. One brake may be paired with one actuator. For example, an actuator may be provided for driving the medical instrument along the insertion axis of the manipulator assembly, and a brake may be provided for inhibiting movement of the medical instrument along the insertion axis.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position LO on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

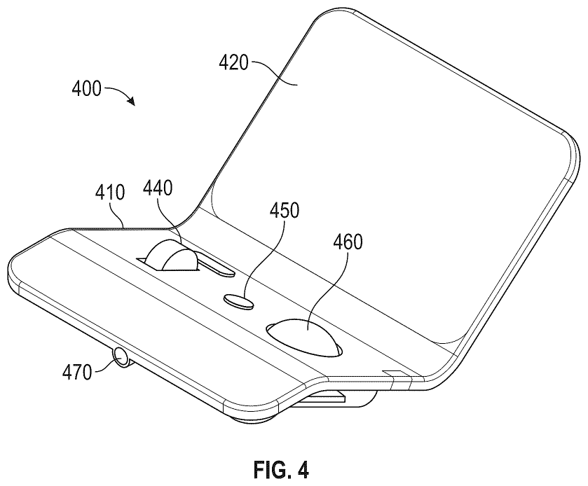

FIG. 4 is a simplified perspective diagram of an input control console 400 according to some embodiments. The input control console 400 may correspond to or may be part of the master assembly 106. A top surface 410 of input control console 400 includes various input controls such as, for example an insertion/retraction control 440, a passive control button 450, a steering control 460 emergency stop button 470. The input control console may further include an integrated display screen, such as screen 420. Although FIG. 4 shows a configuration of the various input controls for an elongate device, it should be understood that input control console 400 can control any variety of instruments and devices and the exact placement, orientation, relative-positioning, and/or the like of the various input controls are exemplary only. It is understood that other configurations of input controls, different numbers of input controls, and/or the like are possible. In some embodiments, input control console 400 is suitable for use as a patient-side input control unit for the elongate device and may, for example, be mounted in proximity to insertion stage 308.

Although not shown in FIG. 4, input control console 400 may optionally include one or more circuit boards, logic boards, and/or the like that are usable to provide power, signal conditioning, interface, and/or other circuitry for input control console 400. In some examples, the one or more circuit boards, logic boards, and/or the like are useable to interface input control console 400 and its various input controls to a control unit for the elongate device. In some examples, the control unit of the elongate device corresponds to the control device of master assembly 106, control system 112, and/or the like. In some examples, the one or more circuit boards, logic boards, and/or the like may include memory and one or more one or more processors, multi-core processors, field programmable gate arrays (FPGAs), application specific integrated circuits (ASICs), and/or the like. In some examples, the memory may include one or more types of machine-readable media. Some common forms of machine-readable media may include floppy disk, flexible disk, hard disk, magnetic tape, any other magnetic medium, CD-ROM, any other optical medium, RAM, PROM, EPROM, FLASH-EPROM, any other memory chip or cartridge, and/or any other medium from which a processor or computer is adapted to read.

In some examples, insertion/retraction control 440 is a single degree of freedom infinite length of travel input control providing infinite length of travel along a first axis usable by the operator to control the insertion depth of the distal end of the elongate device. Insertion/retraction control 440 is depicted as a scroll wheel, however, other types of input controls, including non-infinite length of travel input controls, are possible. In some examples, scrolling of the scroll wheel forward away from the operator increases the insertion depth (insertion) of the distal end of the elongate device and scrolling of the scroll wheel backward toward the operator decreased the insertion depth (retraction) of the distal end of the elongate device. In some examples, insertion/retraction control 440 is usable by the operator to move instrument carriage 306 in and out along insertion stage 308 in order to control the insertion depth of distal end 318.

When insertion/retraction control 440 is an infinite length of travel input control, operating insertion/retraction control 440 in a position-specifying mode allows the operator to exercise precise insertion depth control of the distal end of the elongate device over the full length of travel of the elongate device. In some examples, movement of insertion/retraction control 440 may be detected by the one or more circuit boards, logic boards, and/or the like of input control console 400 using one or more encoders, resolvers, optical sensors, hall effect sensors, and/or the like (not shown). In some examples, feedback applied via one or more electromagnetic actuators, and/or the like may optionally be used to apply haptic feedback to insertion/retraction control 440. In some examples, a scale factor between an amount of movement of insertion/retraction control 440 and an amount of insertion and/or retraction movement by the elongate device is adjustable by the operator and/or control software of the elongate device so that an insertion/retraction velocity of the elongate device relative to an angular velocity of insertion/retraction control may be adjusted to allow both fast insertion and retraction when advantageous and slower more precise insertion and retraction when greater control precision is desired.

In some examples, steering control 460 is a multi-degree of freedom infinite length of travel input control providing infinite length of travel about any number of axes, which in practice may be decomposed into combinations of a left and right rotation, a forward and back rotation, and a spin in place rotation. Steering control 460 is depicted as a track ball, however, other types of input controls, including non-infinite length of travel input controls, are possible. Steering control 460 is usable by the operator to concurrently control both the pitch and yaw of the distal end of the elongate device. In some examples, components of the track ball rotation in the forward and back directions may be used to control a pitch of the distal end of the elongate device and components of the track ball rotation in the left and right directions may be used to control a yaw of the distal end of the elongate device. In some examples, other rotational components of the track ball may be used to control pitch and/or yaw with the operator being optionally able to control whether the direction of rotation is normal and/or inverted relative to the direction applied to the steering (e.g., rotate forward to pitch down and backward to pitch up versus backward to pitch down and forward to pitch up). In some examples, steering control 460 is usable by the operator to manipulate the distances each of the cables extending between the proximal and distal ends of the elongate device are pushed and/or pulled.

In some embodiments, insertion/retraction control 440 and/or steering control 460 include a touch sensor. The touch sensor may be a capacitive touch sensor or any other type of touch sensor. Alternatively or additionally, a pressure sensor may be included. The touch and/or pressure sensor at the input control console 400 may be used to differentiate intended movement by the operator from inadvertent movement due to accidental contact, dropping of input control console 400, and/or the like. Other types of proximity sensors (e.g., ultrasonic sensors, vision sensors, light walls, and/or the like) may be used to detect operator proximity to the input controls. In some examples, one or more wrist detection sensors (e.g., capacitive touch, pressure, and/or similar sensors) in a wrist rest may be used to detect operator proximity to the input controls.

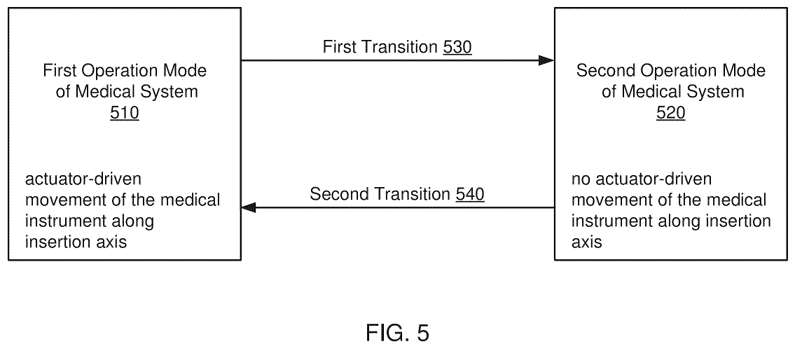

FIG. 5 is an illustration of operation modes of a medical system according to some embodiments. The example shows two different operation modes-a first operation mode 510 and a second operation mode 520. A first transition 530 enables the medical system to transition from operating in the first operation mode 510 to the second operation mode 520, and a second transition 540 enables the medical system to transition from operating in the second operation mode 520 to the first operation mode 510. A medical system may have any number of operation modes, without departing from the disclosure. The first operation mode, the second operation mode, the first transition and the second transition are subsequently discussed.

The first operation mode 510 may be used for operations that involve an actuator driven movement of the medical instrument along a movement axis, e.g., along an insertion axis to move the medical instrument towards or away from target tissue. The first operation mode may be used, for example, when performing a navigation operation. A controller, e.g., based on a servo control loop, may control position and/or velocity of the actuator that causes the movement of the medical instrument along the movement axis. The controller may receive a commanded position and/or velocity and may cause movement according to the commanded position and/or velocity. In the example of a movement along the insertion axis being controlled, a user may operate the insertion/retraction control 440 to provide the commanded position and/or velocity, thereby causing a corresponding movement along the insertion axis by servoing the actuator to follow the commanded position and/or velocity. As previously discussed, the actuator may be equipped with a brake. When in the first operation mode 510, the brake is controlled to be released, allowing actuator driven movement of the medical instrument along the movement axis.

The second operation mode 520 may be used for operations that do not involve an active driving of the actuator to cause movement of the medical instrument along the movement axis. The second operation mode may be used, for example, when conducting a medical operation on the target tissue, e.g., one or more of a biopsy, an ablation, an electroporation, etc. When in the second operation mode 520, the controller that controls the position and/or velocity of the actuator may be configured to hold a current position, e.g., by commanding a constant position and/or zero velocity. The second mode of operation may be associated with system and/or procedural states associated with the user being unlikely to intend actuator driven movement and/or the user being likely to apply external force.

The actuator, while controlled to hold the current position may produce a finite force or torque. Accordingly, an externally applied force or torque, when sufficiently high, could result in a back-driving of the actuator, even while the actuator is being servoed to hold the current position. Specifically referring to the configurations as illustrated in FIGS. 2A, 2B, 2C, 2D, 3A and 3B, when a user interacts with the medical system, back-driving of the medical instrument 304 along the insertion axis may occur under various circumstances, such as when inserting and/or mounting a flexible tool 226 (e.g., an image capture probe, a biopsy tool, etc.) or accidentally contacting the instrument body 312, etc. Such back-driving results in motion of the elongate device 310. For example, when a force in the insertion direction is applied, the elongate device 310 may move slightly in the insertion direction. When the force is no longer applied, the servo-controlled actuator may compensate for the back-driving and cause the actuator to return to the originally held position. This causes the flexible elongate device 310 to move in a retraction direction, back to the original insertion depth. In another example, when a force in the retraction direction is applied, the flexible elongate device 310 may move slightly in the retraction direction. When the force is no longer applied, the servo-controlled actuator may compensate for the back-driving and cause the actuator to return to the originally held position. However, this may not necessarily result in a return of the flexible elongate device 310 back to the original insertion depth. Instead, because of the flexibility in the flexible elongate device 310, the flexible elongate device may be limp, with an incomplete or no return to the original insertion depth. If the system is configured to perform various operations based on detected insertion or retraction of the flexible elongate device 310, then insertion or retraction caused by external force may result in the unintended execution of such operations. One example of such an operation is limp on retract, where force exerted by pull wires within the flexible elongate device 310 is decreased allowing the articulable body portion of the flexible elongate device 310 to lose its pose and/or become more compliant to external forces within the anatomy).

The brake is controlled (e.g., engaged or otherwise applied) to inhibit movement of the medical instrument along the motion axis the second operation mode 520, such as may be caused by an external force. The application of the brake may reduce or eliminate a possible back-driving in the previously described scenarios. The servoing of the actuator may continue while the brake is applied such that the brake and the servoing jointly counteract the back-driving. The brake is controlled (e.g., released or otherwise not applied) to allow actuator driven movement of the medical instrument along the motion axis in the first operation mode 510.

While the above discussion captures the first operation mode 510 and the second operation mode 520 in context of an insertion axis, embodiments of the disclosure are applicable to any other movement axis. For example, the motion axis may include an articulation axis (e.g., pitch or yaw) of the flexible elongate device, or some other motion axis of the flexible elongate device or medical tool.

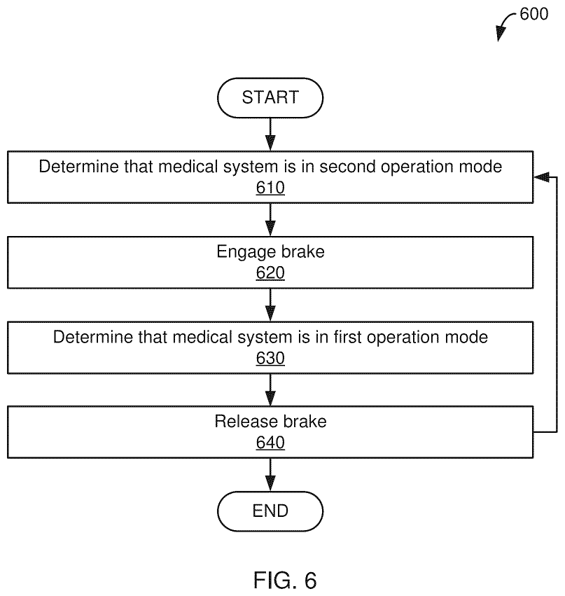

The transitioning between the first and second operation modes, including the engaging and releasing of the brake is subsequently described in reference to the flowchart of FIG. 6. In some embodiments, engagement or release of the brake for motion axis may be triggered based on transitions between the first and second operation modes.

FIG. 6 shows a flowchart of a method 600 for conditional brake engagement to inhibit back-driving of a medical instrument, in accordance with embodiments of the disclosure. On a high level, the method 600 may be used to engage the brake when a user is expected to operate the medical instrument in the second operation mode. The method may further be used to release the brake when a user is expected to operate the medica instrument in the first operation mode.

The method may be implemented using instructions stored on a non-transitory medium that may be executed by a computing system, e.g., the computing system 120.

While the various blocks in FIG. 6 are presented and described sequentially, some or all of the blocks may be executed in different orders, may be combined or omitted, and some or all of the blocks may be executed in parallel. Furthermore, the blocks may be performed actively or passively.

For the purpose of discussing the flowchart, it is assumed that, initially, the medical system is in the first operation mode. This enables an initial transition from the first operation mode to the second operations mode. Alternatively, initially, the medical system may be in the second operation mode. In this case, the initial transition may be from the second operation mode to the first operation mode.

In block 610, a determination is made that the medical system is in the second operation mode. The second operation mode may have been reached or may be reached by a first transition from the first operation mode to the second operation mode. Whether the medical system is operated in the first operation mode or in the second operation mode, in many cases, may be based on user intent. In one example, a user may provide a user input that directly indicates whether the medical system should operate in the first or second operation modes. In other examples, however, sensor data from one or more sensors or other types of user input for operating the medical system may be used to programmatically determine the user intent with respect to operation mode or transitions between operation modes. This allows the user to seamlessly use the medical system without having to devote time or mental/physical resources to select operation modes, while still providing the benefits discussed herein with respect to braking based on operation modes. As such, various factors may indicate whether operation of the medical system should be in the first operation mode or in the second operation mode. Embodiments of the disclosure consider these factors to determine the first transition as subsequently described.

In some embodiments, determining whether the medical system is in the first operation mode (e.g., involving actuator driven movement of the medical instrument along the insertion axis and/or no external force application to the medical instrument) or the second operation mode (e.g., involving no actuator driven movement of the medical instrument along the insertion axis and/or external force application to the medical instrument), which may involve determining transitions between the first and second operation modes, may be performed as follows.

Operation in the second operation mode may be determined based on sensor data obtained from the sensors of the medical system. Various sensors may be used to determine that the user does not intend to drive the instrument along the insertion axis.

In some embodiments, a touch sensor, pressure sensor, or proximity sensor may be used to detect a presence or absence of the user's hand on or near the input device used for controlling the medical instrument along the insertion axis. In some embodiments, operation of the user input device (e.g., operation of a scroll wheel) may be monitored to make the detection. Other sensors may be used without departing from the disclosure.

A detected absence of the user's hand at or near the input device may serve as an indication that the user does not intend to drive the medical instrument along the insertion axis in the near future. Accordingly, the operation of the medical system in the second operation mode may be determined when the sensor(s) detect an absence of touch, proximity, and/or user input for at least a pre-specified time interval, e.g., five seconds.

In some embodiments, a tool recognition sensor may be used to detect a presence or absence of a tool in the medical device. The tool may be a vision probe such as an endoscope-type camera or a tool used to perform a procedure (e.g., a biopsy, ablation, electroporation, etc.).

The vision probe may be used to obtain visual feedback during a navigation operation when driving the medical instrument along the insertion axis. Accordingly, an absence of the vision probe may serve as an indication that the user does not intend to drive the medical instrument along the insertion axis. Thus, the operation of the medical system in the second operation mode may be determined when the tool recognition sensor detects a removal of the vision probe.

Tools used to perform a procedure benefit from a mechanically steady operating environment. Accordingly, the use of such tools may serve as an indication that the user does not intend to drive the medical instrument along the insertion axis. Thus, the operation of the medical system in the second operation mode may be determined when the tool recognition sensor detects an insertion of a tool to be used for performing a procedure. In some embodiments the type of the tool is considered when determining the operation mode. For a first type of tool, the second operation mode is determined. A biopsy needle is an example of a first type of tool that benefits from engaging the brake. For a second type of tool, the second operation mode is not determined. A forceps is an example of a second type of tool that does not require an engagement of the brake.

In some embodiments, a force sensor may be used to detect the presence of an external force that could result in movement of the medical instrument along the insertion axis. Such an external force may be associated with an insertion or removal of a tool, or with a touch, a collision, etc. An external force may also be detected based on a control error associated with the actuator when driving the instrument. For example, a control error may be detected by comparing an actual position of the actuator (obtained, for example, from an encoder) with the desired position of the actuator. To counter unintended movement along the insertion axis in presence of an external force, the operation of the medical system in the second operation mode may be determined.

In some embodiments, a proximity of the medical instrument from the target, e.g., target tissue, is used to determine the second operation mode. Specifically, after performing a registration as previously described, and using the sensors of the medical system to track the location of the medical instrument, the distance of the medical instrument from the target can be determined. Once a certain proximity is reached, the actual medical procedure may be performed using the medical instrument. Accordingly, once a certain proximity is reached, the second operation mode may be determined.

In some embodiments the second operation mode may be determined based on a user request. For example, a user may provide a control input explicitly specifying that the system is to be operated in the second operation mode.

In block 620, based on the determination that the medical system is in the second operation mode, the brake is engaged to inhibit movement of the medical instrument along the insertion axis caused by an external force. While the brake is engaged, the actuator may still be servoed at the current position. Accordingly, the brake and the servo-controlled actuator jointly counteract any actual or possible back-driving.

In block 630, a determination is made that the medical system is operating in the first operation mode. The first operation mode may have been reached or may be reached by a second transition from the second operation mode to the first operation mode.

In some embodiments, determining that the medical system is in the first operation mode involving actuator driven movement of the medical instrument along the insertion axis may be performed as follows.

The first operation mode may be determined based on sensor data obtained from the sensors of the medical system. Various sensors may be used to determine that the user intends to drive the instrument along the insertion axis.

As previously discussed, in some embodiments, a touch sensor, pressure sensor, proximity sensor, and/or other sensor may be used to detect a presence or absence of the user's hand on or near the input device used for controlling the medical instrument along the insertion axis.

A detected presence of the user's hand at or near the input device may serve as an indication that the user intends to drive the medical instrument along the insertion axis in the near future. Accordingly, the first operation mode may be determined when the sensor(s) detect a presence of touch, proximity, and/or user input. A detection may immediately trigger the determination of the first operation mode, without delay. Further, the detection may override other potentially contradicting detections. For example, while an absence of a vision probe, viewed in isolation, may serve as an indication that the user does not intend to drive the medical instrument along the insertion axis, the detection of the user's hand at or near the input device would result in the determination of the first operation mode, to ensure responsiveness of the medical system to a user input, even in absence of the vision probe.

As previously discussed, in some embodiments, a tool recognition sensor may be used to detect a presence or absence of a tool in the medical device. The tool may be a vision probe such as an endoscope-type camera or a tool used to perform a procedure (e.g., a biopsy, ablation, electroporation, etc.).

The vision probe may be used to obtain visual feedback during a navigation operation when driving the medical instrument along the insertion axis. Accordingly, a presence of the vision probe may serve as an indication that the user intends to drive the medical instrument along the insertion axis. Thus, the first operation mode may be determined when the tool recognition sensor detects an insertion of the vision probe.

Tools used to perform a procedure benefit from a mechanically steady operating environment. Removal of the tool may suggest that that the user intends to drive the medical instrument along the insertion axis, e.g., to move from one target site to another target site. Thus, the first operation mode may be determined when the tool recognition sensor detects a removal of a tool for performing a procedure.

In some embodiments the first operation mode may be determined based on a user request. For example, a user may provide a control input explicitly specifying that the system is to be operated in the first operation mode.

In block 640, based on the determination that the medical system is in the first operation mode, the brake is released to allow movement of the medical instrument along the insertion axis.

The method 600 may be executed in a loop that enables repeated transitions between the first and second operation modes. The medical system may remain in the first and second operation modes for any amount of time, until a first or second transition occurs. However, by monitoring current conditions as described, the engagement and release of the brake is not directly coupled to an actual absence or presence of an actuator-driven movement, which could result in a frequent and excessive engagement and release of the brake. Instead, based on the operations as described, the driving and the absence of driving in the foreseeable future are predicted as states, thereby limiting the cycling of the brake between engaged and released to a lower frequency, while ensuring that spontaneous driving is possible, when needed. Further, while a number of factors that may determine operation in the first vs the second operation modes have been discussed, other factors may be considered as well. For example, the likeliness of undesirable back-driving may be affected by actuator size, mechanical design, and/or current mechanical or kinematic configuration. Specifically, for example, gravity may further increase the likelihood of back-driving, if the back-driving occurs in a non-horizontal direction, merely based on the additional weight that needs to be supported by the actuator being servoed. Accordingly, the engaging of the brake may be necessary to avoid back-driving when the insertion axis is in a vertical direction or non-horizontal direction, but not to avoid back-driving when the insertion axis is in a horizontal direction.

While the method 600 is described in context of an insertion axis, the method is equally applicable to other movement axes. For example, the method may be applied to a manipulator arm with multiple joints, where both insertion and elevation may be adjusted during driving of a flexible elongate device in an insertion/retraction direction.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM), a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A medical system comprising:
a manipulator assembly comprising:
an actuator for driving a medical instrument along an insertion axis of the manipulator assembly; and
a brake for inhibiting movement of the medical instrument along the insertion axis; and
a control system coupled to the manipulator assembly, the control system configured to:
determine whether the medical system is in a first operation mode involving actuator driven movement of the medical instrument along the insertion axis or a second operation mode involving no actuator-driven movement of the medical instrument along the insertion axis; and
based determining that the medical system is in the second operation mode, control the brake to inhibit movement of the medical instrument along the insertion axis caused by an external force.

2. The medical system of claim 1, wherein the control system is further configured to:
based on determining that the medical system is in the first operation mode, control the brake to allow the actuator-driven movement of the medical instrument along the insertion axis.

3. The medical system of claim 1, further comprising one or more sensors configured to generate sensor data regarding the medical system, and wherein the control system determines whether the medical system is in the first operation mode or the second operation mode based on the sensor data.

4. The medical system of claim 3, further comprising:
a user input device for controlling the driving of the medical instrument along the insertion axis based on a user input,
wherein the one or more sensors comprise a touch sensor disposed on the user input device.

5. The medical system of claim 4, wherein the control system determines that the medical system is in the first operation mode based on a detection of a touch by the touch sensor.

6. The medical system of claim 4, wherein the control system determines that the medical system is in the second operation mode based on a detection of an absence of touch by the touch sensor for at least a pre-specified time interval.

7. The medical system of claim 4, wherein the touch sensor comprises a capacitive touch sensor.

8. The medical system of claim 4, wherein the control system determines that the medical system is in the first operation mode based on a detection of a user input by the user input device.

9. The medical system of claim 4, wherein the control system determines that the medical system is in the second operation mode based on a detection of an absence of a user input by the user input device for at least a pre-specified time interval.

10. The medical system of claim 3, wherein the medical instrument comprises a flexible elongate device with a lumen configured to receive a flexible tool, and the one or more sensors comprise a tool recognition sensor configured to detect a presence of the flexible tool in the lumen.

11. The medical system of claim 10, wherein the control system determines that the medical system is in the first operation mode based on a presence of the flexible tool.

12. The medical system of claim 10, wherein the flexible tool is a vision probe.

13. The medical system of claim 10, wherein the tool recognition sensor comprises one selected from a group consisting of an inductive sensor, a capacitive sensor, an optical sensor, and a magnetic switch.

14. The medical system of claim 3, wherein the one or more sensors comprise a force sensor configured to sense the external force.

15. The medical system of claim 14, wherein the control system determines that the medical system is in the second operation mode based on a detection of a presence of the external force.

16. The medical system of claim 1, wherein the external force is detected based on a control error associated with the actuator when driving the medical instrument.

17. The medical system of claim 2, wherein the control system determines that the medical system is in the first operation mode or the second operation mode based on a proximity of the medical instrument to a target.

18. The medical system of claim 2, wherein the control system determines that the medical system is in the first operation mode or the second operation mode based on a user request.

19. The medical system of claim 1, wherein when in the second operation mode, the control system is configured to servo the actuator while the brake is engaged.

20. The medical system of claim 1, wherein:
the first operation mode is associated with a navigation operation comprising movement of the medical instrument within an anatomical passageway toward a target tissue, and the second operation mode is associated with a medical operation conducted on the target tissue.

21. The medical system of claim 20, wherein the medical operation comprises one or more of a biopsy, an ablation, and an electroporation.

22. A non-transitory machine-readable medium comprising a plurality of machine-readable instructions executed by one or more processors associated with a medical system, the plurality of machine-readable instructions causing the one or more processors to perform a method comprising:
determining whether the medical system is in a first operation mode involving actuator driven movement of a medical instrument along an insertion axis or a second operation mode involving no actuator-driven movement of the medical instrument along the insertion axis; and
based determining that the medical system is in the second operation mode, controlling a brake to inhibit movement of the medical instrument along the insertion axis caused by an external force.

23-42. (canceled)

43. A method for operating a medical system, comprising:
determining whether the medical system is in a first operation mode involving actuator-driven movement of a medical instrument along an insertion axis or a second operation mode involving no actuator driven movement of the medical instrument along the insertion axis; and
based determining that the medical system is in the second operation mode, controlling a brake to inhibit movement of the medical instrument along the insertion axis caused by an external force.

44. The method of claim 43, further comprising:
based on determining that the medical system is in the first operation mode, controlling the brake to allow the actuator-driven movement of the medical instrument along the insertion axis.

45. The method of claim 43, wherein the external force is detected based on a control error associated with the actuator when driving the medical instrument.
